SEQUENCE LISTING (6 sequences)

SEQ ID NO: 1 - DNA, 38 bp, Artificial Sequence
  Description of Artificial Sequence Primer
gtggcccaggcggccctgactcagccgtcctcggtgtc

SEQ ID NO: 2 - DNA, 34 bp, Artificial Sequence
  Description of Artificial Sequence Primer
cgaagatctagaggactgacctaggacggtcagg

SEQ ID NO: 3 - DNA, 39 bp, Artificial Sequence
  Description of Artificial Sequence Primer
ggtcagtcctctagatcttccgccgtgacgttggacgag

SEQ ID NO: 4 - DNA, 44 bp, Artificial Sequence
  Description of Artificial Sequence Primer
ctggccggcctggccactagtggaggagacgatgacttcggtcc

SEQ ID NO: 5 - DNA, 42 bp, Artificial Sequence
  Description of Artificial Sequence Primer
gaggaggaggaggaggaggtggcccaggcggccgtgactcag

SEQ ID NO: 6 - DNA, 47 bp, Artificial Sequence
  Description of Artificial Sequence Primer
gaggaggaggaggaggaggagctggccggcctggccactagtggagg

(19) United States
(12) Patent Application Publication
(10) Pub. No.: US 2002/0058285 A1
(43) Pub. Date: May 16, 2002

(54) Method for the identification of substances mimicking mammal epitopes

(76) Inventors: Hans-Georg Frank, Aachen (DE); Peter Kaufmann, Aachen (DE)

Correspondence Address: JACOBSON HOLMAN PLLC, 400 SEVENTH STREET N.W., SUITE 600, WASHINGTON, DC, 20004, US

(21) Appl. No.: 09/750,021

(22) Filed: Dec. 29, 2000

(30) Foreign Application Priority Data
Dec. 30, 1999 (DE) 199 64 046.7

(51) Int. Cl. G01N 33/53; A61K 38/08; A61K 38/10

(52) U.S. Cl. 435/7.1; 514/14; 514/15; 514/16; 530/327; 530/328; 530/329

(57) ABSTRACT

A method for the preparation of specific monoclonal immunological binding molecules having binding capacity to mammal epitopes, comprising the following steps:

a) isolation of structures containing said epitopes to obtain an epitope preparation;

b) immunization of non-mammals with the epitope preparation to obtain an immune response;

c) immortalization of the immune response to obtain a library of immunological binding molecules;

d) selection of the immunological binding molecules by means of the epitopes to obtain specific monoclonal immunological binding molecules.

The specific monoclonal immunological binding molecules obtained can be used for identifying, in a library of low molecular weight substances, those having a high binding affinity, thus mimicking the epitopes of mammals.

The present invention relates to a method for the preparation of specific monoclonal immunological binding molecules having binding capacity to mammal epitopes, the immunological binding molecules, and a method for the identification of low molecular weight substances mimicking mammal epitopes, and the corresponding low molecular weight substances.

Methods for the preparation of immunological binding molecules, especially antibodies, are known to those skilled in the art. Common methods are based on the immunization of host animals with substances against which antibodies are to be raised. To enhance the immune response, adjuvants containing highly potent immunogens are usually added. The immune response achieved in the host animal by one or more immunizations can be used in the form of polyclonal antibody sera or immortalized by hybridoma technology, optionally followed by monoclonalization through isolation of single cells.

Alternatively, the genetic information contained in the cells relating to the structure of antibodies can be used to obtain synthetic binding molecules, such as single-chain antibodies (scFv).

All these methods rely on the original immunization of the host animal and therefore depend on the quality of the immune response produced thereby. It is to be taken into account that the animals used for immunization when immortalization of the immune response using hybridoma technology is intended are almost exclusively mice since appropriate immortalized myeloma cells are available only for laboratory rodents.

In the usual immunization of mice or rabbits, good immune responses are achieved only when the corresponding protein is recognized as “foreign” by the host animal. This is a problem, in particular, when the relevant structures are structures which were conserved over a long stretch of evolution.

In many cases, it is of particular interest to establish also the structures of the substances acting as immunogens. For example, when they are simple peptides and proteins, they can be identified by means of the immunological binding molecules obtained and isolated and characterized by chromatographical methods. The exact structural elucidation is subsequently effected, for example, by protein sequencing.

However, when the immunogenic structures are compounds having a complex structure, e.g., because lipid structures are involved, characterization is more difficult because compounds like these often lose their structure when purified and thus are no longer recognized by the immunological binding molecules. In such a case, substances having identical three-dimensional structures would be of great interest since such substances could possibly be used as components of vaccines to achieve immunization.

It has been the object of the present invention to overcome the mentioned problems and drawbacks of the prior art.

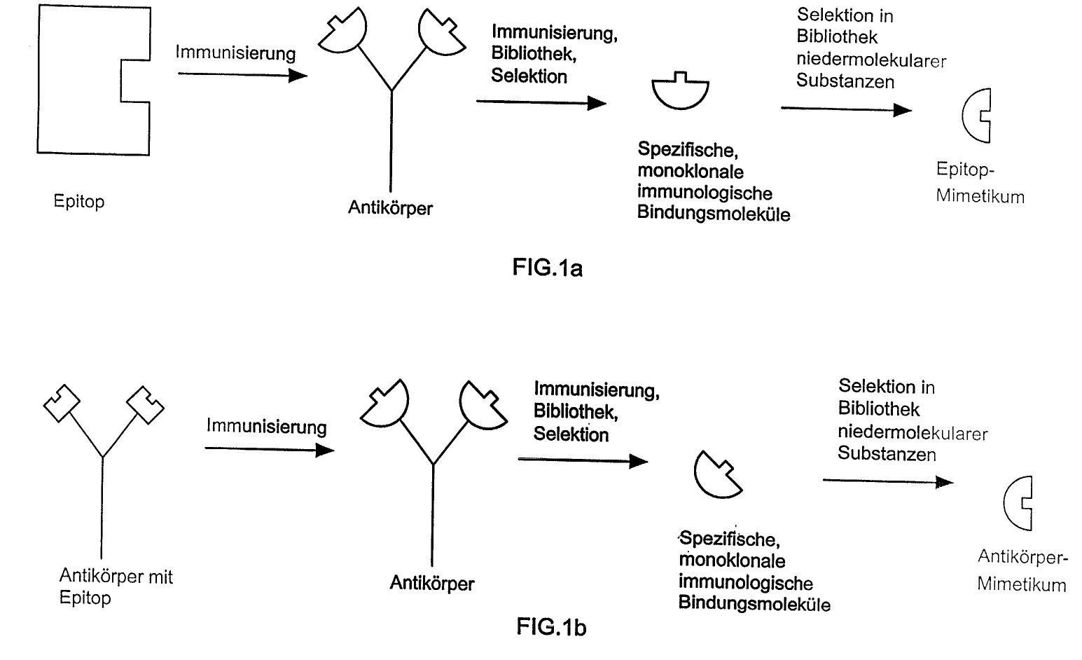

FIG. 1 is a schematic illustration of the method according to the invention.

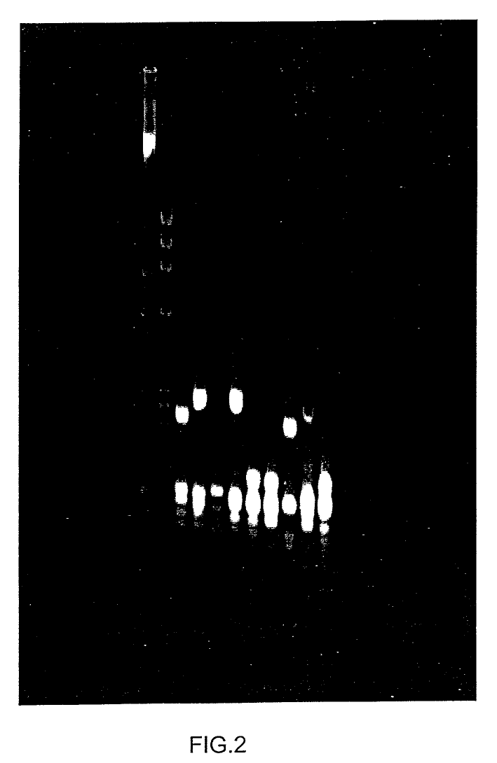

FIG. 2 shows an agarose gel of the inserts of some members of a phage library.

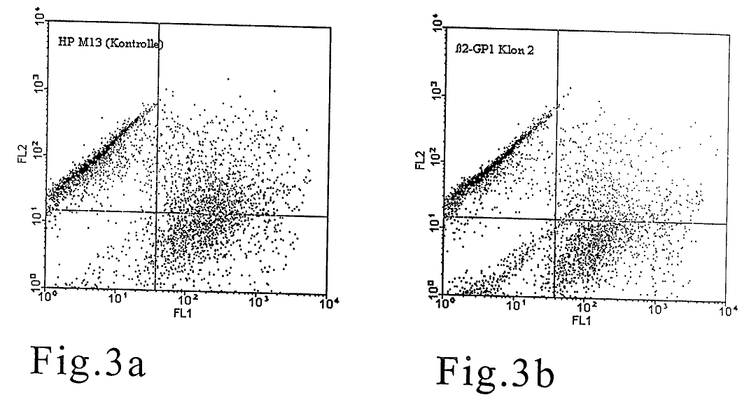

FIG. 3 shows diagrams for the evaluation of a flow-cytometric experiment.

In one aspect of the invention, the object is achieved by a method for the preparation of specific monoclonal immunological binding molecules having binding capacity to mammal epitopes, comprising the following steps:

a) isolation of structures containing said epitopes to obtain an epitope preparation;

b) immunization of non-mammals with the epitope preparation to obtain an immune response;

c) immortalization of the immune response to obtain a library of immunological binding molecules;

d) selection of the immunological binding molecules by means of the epitopes to obtain specific monoclonal immunological binding molecules.

An “immunological binding molecule” as used herein means a molecule which is directly or indirectly obtained by immunization and can bind to other molecules. In particular, the term encompasses antibodies and antibody fragments as well as single-chain antibodies.

“Specific immunological binding molecules” means that these binding molecules will bind to other substances with a dissociation constant, K_{d}, of less than 10⁻⁵, preferably between 10⁻⁶ and 10⁻¹², mol/l.

“Monoclonal immunological binding molecules” are those which can be produced by a plurality of cells, but wherein all cells express identical binding molecules. “Immortalization of the immune response” means that the immune response obtained is converted to a form which allows to have corresponding binding molecules produced by cells in vitro over an extended period of time.

A “library of immunological binding molecules” means a plurality of different binding molecules which are still connected to their DNA information in such a way that monoclonal binding molecules can be obtained by isolating single members of the library, especially libraries of single-chain antibodies in phage libraries.

“Selection of immunological binding molecules” means a method by which the binding capacity of the binding molecules to substances is tested, and molecules exhibiting high binding affinities are isolated.

In this method, epitope preparations are produced in a first step. In principle, any epitope is suitable, but preferred are epitopes expressed on the surfaces of cells, especially of trophoblasts or tumor cells, or epitopes involved in cell-virus fusion, or epitopes of endogenous antibodies. The structures thus obtained are used for the immunization of non-mammals, optionally adding usual adjuvants. After an immune response has been produced, this immune response is immortalized, i.e., the nucleic acid sequences of the non-mammal responsible for the immune response are transferred to a system in which the immune response can be utilized by expressing the nucleic acid information.

One particular preferred method for this is conversion of the nucleic acid sequence, using polymerase chain reaction or similar systems, to an scFv fragment which is then coupled to a phage to obtain a phage library. The thus obtained phage library contains a complete spectrum of all antibody structures present in the non-mammal and, in particular, formed by the immunization.

Since only a low number of the immunological binding molecules contained in the library actually possess a high affinity for the desired epitopes, it is necessary that at least one selection step follow by which the high affinity binding molecules can be enriched. This can be effected, for example, by fixing the epitope structures to a solid phase with which the entire library of immunological binding molecules is incubated to wash off the low affinity binding molecules by washing steps. The remaining high affinity binding molecules can subsequently be converted to monoclonal specific immunological binding molecules by isolation of single cells. Corresponding techniques are known to those skilled in the art as “panning” and described, for example, in Kay B. K., Winter J., McCafferty J. (1996) “Phage display of peptides and proteins: a laboratory manual; Academic Press, San Diego.

In a preferred embodiment, the employed structures containing the epitopes are also antibodies so that anti-idiotypic binding molecules can be obtained in this way. Of course, the corresponding antibodies or antibody fragments also may have been obtained by the method according to the invention.

The method is useful, in particular, for conserved epitopes. “Conserved epitopes” of mammals are especially those which

a) occur in different mammal species; and

b) have relatively few interspecies differences; and optionally

c) are characterized in that consensus sequences or consensus structures can be defined at least for the mammals, c) applying only for epitopes of which sequence data can be established due to their type of material.

Conserved mammal epitopes have a higher immunogenicity in non-mammals than they have in mammals.

Herein, the term “conserved epitopes” is intended to encompass, in addition to this meaning derived from phylogenesis, also all ontogenetically relevant human epitopes which

a) are expressed in human trophoblastic tissue, in human embryonic tissue, and/or in malignant degenerate cells and tissues or intermediate stages between normal and malignant degenerate tissue; and

b) are not expressed in the normal adult cells and tissues which are differentiated in a way typical of that tissue and which correspond to said malignant degenerate cells; and

c) are characterized by exhibiting, as a rule, a lower immunogenicity in mammals than they exhibit in non-mammals which lack both trophoblast and placenta; and

d) are characterized by being important as viral envelope and fusion proteins in virus-cell fusion or cell-cell fusion of the trophoblast, or by being important to cell-cell fusion, or by being derived from genes for viral fusion proteins integrated into the respective mammal genome during phylogenesis (so-called endogenous retroviral genes, ERV).

The method according to the invention is useful, in particular, for epitopes which cannot be prepared or are difficult to prepare biochemically or by molecular biology, especially epitopes which are formed or exposed by a conformational change upon ligand binding to membrane proteins, complex epitopes formed by the association of subunits, or epitopes formed by the association of substances from different classes of substances, e.g., complexes of membrane lipids or membrane proteins.

Regarding structure, all epitopes are suitable, especially proteins, nucleic acids, lipids, carbohydrates and combinations of these groups of substances.

Further epitopes which could be of particular value to the method according to the invention include:

a) epitopes formed by oligomeric to polymeric carbohydrates, irrespective of whether they exist by themselves or occur in association with proteins, lipids or nucleic acids;

b) epitopes formed by the association of subunits at the surface of cells, especially receptor molecules as well as virus-cell, cell-cell and cell-matrix contact molecules.

The preferred species for immunization include birds, especially chickens, and amphibians, especially Xenopus laevis.

The invention also relates to the immunological binding molecules which can be obtained by the method according to the invention, and a diagnostic agent containing the immunological binding molecules.

A further aspect of the invention is a method for the identification of low molecular weight substances mimicking mammal epitopes, wherein the specific monoclonal immunological binding molecules obtained by the method described above are used for identifying, in a library of low molecular weight substances, those having a high binding affinity for the immunological binding molecules. Thus, appropriate molecules mimic the native structure of the originally employed epitopes, but can be selected from basically different classes of substances; they have similar stereochemical properties. In the following, they are sometimes referred to as mimetics.

The method according to the invention is further illustrated with reference to the scheme in FIG. 1a. Immunization yields antibodies directed against an epitope. The structures relevant to epitope recognition are enriched in a library by immortalization and selected. These binding molecules can be used for identifying, in a library of low molecular weight substances, a low molecular weight substance which has the same binding properties as the original epitope despite its having a different chemical structure. It is referred to as a mimetic of the epitope.

FIG. 1b shows a special case of the method according to the invention wherein an antibody is employed as the structure bearing the epitope. By the same methods, mimetics are obtained which now mimic the structure of the originally employed antibody.

“Low molecular weight substances” are aliphatic and/or aromatic structures having up to 200 carbon atoms which may optionally have one or more functional groups such as hydroxy, carbonyl, carboxy functions, ester, ether, amine, amide, phosphate, sulfate functions, preferably peptides comprising up to 50 amino acids, preferably less than 30 amino acids.

The preferred mammal species the epitopes of which are of interest is Homo sapiens. However, as further explained below, epitopes from pets, such as cats and dogs, or pests, such as rats or mice, can also be of particular interest.

Said library of low molecular weight substances preferably contains peptides, especially in the form of a phage peptide library. Preferably, the peptides comprise from 7 to 15 amino acids, more preferably 9 or more and 12 or less amino acids.

The invention also relates to low molecular weight substances which can be identified by the method according to the invention for the identification of the low molecular weight substances, and a medicament containing at least one of the low molecular weight substances according to the invention.

The use of the methods and substances according to the invention is further illustrated by the following possible applications without being limited thereto.

WO-A-93/06857 already describes a vaccine for contraception in which a protein composition purified from trophoblast membranes is used as a vaccine. Due to the low immunogenicity of human epitopes in humans, little success is to be expected from this.

In humans, the early embryo invasively implants itself as a blastocyst in the stromal portion of the endometrium, i.e., the blastocyst penetrates the uterine epithelium, which results in interstitial implantation. The penetrating cell population is the outer, epithelial stratum of cells, the so-called trophoblast. Interstitial implantation occurs in humans and in a number of related mammals, such as mice and rats. During the whole pregnancy, single trophoblast cells penetrate the maternal tissues to within the region of the endometrial/myometrial transition zone, penetrate the wall of maternal arteries and replace the endothelium there. When penetrating the uterine epithelium, the cells of the trophoblast fuse to a syncytial aggregate, a process which is indispensable to a successful implantation. The process of trophoblastic syncytiogenesis is triggered by signal epitopes of which it is only known to date that a flip of phosphatidylserine to the exterior of the plasma membrane is involved in their generation. Thus, it is known that

a) women having an increased anti-phospholipid antibody level (e.g., in lupus erythematodes) have more problems during pregnancy and are in part infertile;

b) the externalization of phosphatidylserine to the exterior of the plasma membrane immediately precedes the syncytial fusion;

c) antibodies induced against phosphatidylserine in test animals can inhibit syncytial fusion in vitro.

The flip of phosphatidylserine is not a trophoblast-specific phenomenon, but an event occurring in the body during each apoptosis. Apoptopic cells fuse rarely, and if so, they do it always with similar cells. Therefore, there are postulated further epitopes which may be associated with phosphatidylserine and which trigger the tissue-specific event of syncytial fusion only in trophoblasts, in the genesis of skeletal muscular fibers, and in osteoclasts. Since these epitopes formed with the participation of phosphatidylserine are not pure protein epitopes, they are difficult to isolate and characterize biochemically.

Using the method according to the invention, substances can be isolated which are suitable as vaccines for inhibiting syncytial fusion. Thus, for example, trophoblast preparations may be used to immunize chickens. Since chickens have neither trophoblasts nor trophoblast implantations and consequently have a totally different reproduction, which is based on the laying of fertilized eggs, they are capable of producing a sufficient immune response thereto. Then, the immune response can be immortalized as a phage library, for example, using the phage display technology. From these phage libraries, those single-chain antibodies are isolated which specifically bind to trophoblast cells. As an alternative or additional selection step, there may also be selected those single-chain antibodies which can inhibit the syncytial fusion. The thus isolated antibody structures can in turn be used for isolating substances which mimic the structure of trophoblast epitopes. For example, substance libraries, such as peptide libraries, which may optionally also be present as phage display libraries, can be screened. Since the thus obtained peptides are “mimics” of the original epitopes, they may form the base for the development of vaccines which, after antibody production in the mammal, again have an inhibiting effect on the syncytial fusion and thus exhibit contraceptive activity.

In addition to being applied to humans, this contraceptive activity may also be used to particular advantage for application to pets for non-surgical contraception and for the control of pests using contraceptive eatable baits.

From the principles set forth above, the following three contraceptive applications of the method according to the invention or of the obtained substances can be realized:

### I. Contraception

a) The fusion of the trophoblast cells does not occur already during the intrauterine passage, but only during implantation, and the delay of syncytial fusion is achieved by IgA antibodies against phosphatidylserine released into the uterine secretion. Using the low molecular weight substances obtained by the method according to the invention, these natural IgA antibodies could be neutralized. The premature syncytial fusion would abruptly stop the growth of the blastocyst.

b) Although contraceptive antibodies are not suitable for long-term contraception due to the necessity of intravenous application and possible immunological reactions, these antibodies could be employed as epitope-bearing structures. However, using the method according to the invention, low molecular weight substances can be identified which have the same binding capacity as these antibodies and are suitable for contraception due to their low molecular weight.

### II. Fertility Impairment

In the so-called aPL syndrome, antibodies directed against phosphatidylserine are produced which cause a broad range of clinical symptoms from clotting disorders to pregnancy disorders to infertility. These phosphatidylserine-recognizing antibodies (PS antibodies) recognize structures involved in syncytial fusion. Since the aPL syndrome exists in differently pronounced forms, it is to be considered that mimetics selected by the method according to the invention against the antibodies inhibiting syncytial fusion are suitable, on the one hand, for the diagnosis and classification of aPL syndrome and, on the other hand, for the topical or systemic administration of the mimetics by blocking the natural PS antibodies for treatment of the aPL syndrome.

### III. Tumor Diagnostics and Therapy

In addition, the methods and compounds according to the invention can also be employed in tumor diagnostics and treatment. The above described transmigration of tissue by epithelial cells having detached from their basal membrane can be observed, apart from pregnancy, only in malignant tumors, especially in the carcinomas derived from epithelial and glandular tissues.

The invasion of malignant degenerate epithelial cells has its ontogenetic precursors in the invading trophoblast cell. Therefore, both invasive situations have similar properties:

a) Although malignant degenerate cells are genetically and phenotypically different from the normal body cells, there is no clinically effective immune response against these cells. Similarly, a human trophoblast is not rejected, although it is even an allohaploid transplant, formally. In both processes, the expected T-cell mediated immune response does not occur.

b) Many molecules which are expressed in the trophoblast during pregnancy do no longer appear in normal adult tissues. However, they may be reexpressed during malignant degeneration. Those epitopes which are expressed both during the fetal period and during carcinogenesis are designated as oncofetal epitopes. Due to the extended analogies between trophoblastic invasion and tumor invasion, there is a wide field of application for the immunological binding molecules according to the invention and the low molecular weight substance in the field of tumor diagnostics and tumor therapy.

### IV. Anti-Infective Agents

In the virus-cell fusion, intermediary lipid protein complexes occur at the cell surface which are similar to those formed in syncytial fusion. The membranes of adenoviruses and retroviruses originate from the infected cell and are enriched in fusion proteins during extrusion from the cell; these fusion proteins are highly conserved within each group of viruses. During infections of a new cell, fusion with the plasmalemma occurs with herpes viruses and HIV, an event which is similar to cell/cell fusion. In the syncytial fusion of the trophoblast, an envelope protein (syncytin) of human endogenous viruses is centrally involved (Mi S., Lee X., Li X., Veldman G. M., Finnerty H., Racie L., LaVallie E., Tang X. Y., Edouard P., Howes S., Keith J. C. Jr., McCoy J. M. (2000), “Syncytin is a captive retroviral envelope protein involved in human placental morphogenesis”, Nature 403: 785-789). In viral infectious diseases (e.g., cytomegalovirus, hepatitis C, HIV), PS antibodies can also occur, but usually only for a short time. By the method according to the invention for producing specific binding molecules for the epitopes involved in the fusion, there can be obtained mimetics suitable as vaccines. Further, through the use of the thus obtained antibodies as epitopes in the method according to the invention, low molecular mimetics could be obtained which have the binding properties of the fusion-inhibiting antibodies and thus can be used as anti-infective agents.

In the case of generalized, life-threatening viral infections, the antibodies against virus-cell fusion contained according to the invention may also be employed for adoptive immunization.

### EXAMPLE

The following embodiments are illustrative of the application of the method according to the invention. The example comprises the steps of immunization of a non-mammal with different antigen preparations, the establishing of a phage library, and the selection of specific immunological binding molecules as intended in the method.

### Immunization of the Animals

Twelve white leghorn chickens aged from 6 to 18 months were immunized. The immunization protocols were based on published standard schemes (Gassmann, M. et al., FASEB J. (1990) 4: 2528-2532) and comprised a first injection of the antigen preparation (see Table 2) in complete Freund's adjuvant, followed by two booster injections at intervals of 2 to 4 weeks each. The booster injections were performed with incomplete Freund's adjuvant. When the immunization was effected with living human cells, a million cells was administered per injection with no adjuvant (see also Table 2). Five days after the last injection, the immunized animals were sacrificed, the spleens were removed and transferred to the laboratory in a sterile isotonic solution.

### Preparation of Splenocytes, Total RNA and cDNA

Under sterile conditions, the spleen was cut into 8-10 smaller pieces. These pieces were further carefully suspended manually in 3 ml of sterile isotonic saline in an Elvehjem glass tube using the appropriate pestle. The resulting suspension was filtrated through a stainless steel sieve (150 mesh), the splenocytes contained in the filtrate were centrifuged to a pellet (400 x g, 5 min at room temperature), followed by selectively lysing the erythrocytes in lysis buffer (0.15 M NH₄Cl; 1 mM KHCO₃; 0.1 mM Na₂EDTA; pH 7.2). From the remaining splenocytes, the total RNA was prepared, and cDNA was produced with oligo-dT primers after reverse transcription.

### Polymerase Chain Reactions (PCR) for Establishing Libraries

Primers which can be used for amplifying the variable regions of the light (Vk) and heavy chains (Vh) of the immunoglobulin cDNAs are summarized in Table 1 (Andris-Widhopf, I., Rader, C., Steinberger, P., Fuller, R., Barbas, C. F., III (2000), Methods for the generation of chicken monoclonal antibody fragments by phage display, J. Immunol. Methods 242, 159-181). The PCR was performed with the following parameters: The initial denaturation was effected at 94° C. for 1 min, followed by 30 cycles with 15 s of denaturation (94° C.), 15 s of annealing (56° C.) and 90 s of elongation (74° C.). The primers introduce an overlap region which is required for splice overlap extension PCR in the establishing of the segments coding for scFv. The PCR yields products with sizes of about 350 bp. After being thoroughly purified, the products were employed for splice overlap extension PCR.

### Splice Overlap Extension PCR

Equimolar amounts (100 ng each) of the amplificates of Vk and Vh were employed in splice overlap extension PCR. The reaction mixtures further contained a pair of specifically defined primers (see Table 1; Andris-Widhopf, J., Rader, C., Steinberger, P., Fuller, R., Barbas, C. F., III (2000), Methods for the generation of chicken monoclonal antibody fragments by phage display, J. Immunol. Methods 242, 159-181) which introduce the restriction sites into the product, which are required, inter alia, for restriction and ligation. Initial denaturation at 94° C. for 1 min was followed by 35 cycles with 15 s of denaturation (94° C.), 15 s of annealing (56° C.) and 120 s of elongation (74° C.). The desired product has a size of about 750 bp.

### Vector and Ligation

The vector used was pComb3H-SS (Barbas, C. F., 3rd. Curr. Opin. Biotechnol. (1993) 4: 526-530, Biassoni, R. et al., Semin. Cancer Biol. (1999) 9: 13-18, Siegel, D. L. et al., J. Immunol. Methods (1997) 206: 73-85, Andris-Widhopf, J., Rader, C., Steinberger, P., Fuller, R., Barbas, C. F., III (2000), Methods for the generation of chicken monoclonal antibody fragments by phage display, J. Immunol. Methods 242, 159-181).

This vector and the above described primers were especially prepared and designed for the phage display of antibody libraries in the Scripps Research Institute, La Jolla, Calif., USA.

Prior to the ligation, the vector was linearized by digestion with SfiI. Since the vector contains two asymmetric cleaving sites of this restriction enzyme, directional cloning is directly possible after digestion and purification of the vector without an additional digestion step. At the same time, the product of the splice overlap extension PCR was also digested with the enzyme, and the restricted product was purified.

### Establishing of the Phage Antibody Libraries

The prepared vector (14 μg) and the PCR product (10 μg) were incubated together in the presence of 20 units T4 ligase over night at 4° C., the reaction products were precipitated with ethanol and resuspended in 50 μl of bidistilled water. This DNA solution was used to transform electrocompetent E. coli (XL-1 Blue). The number of transformants was determined by titration on LB ampicillin plates (Sambrook, J., Fritsch, E. F. and Maniatis, T., Cold Spring Harbor Laboratory (1989), Cold Spring Harbor, N.Y.), and the phage libraries were harvested after superinfection of the transformed E. coli with the helper phage M13KO7. The libraries in the phage form were stored both individually and as a mixed total library (cumulative library).

In order to get an impression of the diversity of the library and to exclude a dominance of just a few clones, the band patterns of 9 arbitrarily selected clones were analyzed after restriction of the insert (see FIG. 1). For checking the diversity of the cumulative library, 9 clones were arbitrarily picked, the plasmids were isolated and the inserts amplified in a PCR. The PCR products were purified, restricted with MspI, separated in a 2% agarose gel and stained with ethidium bromide. The sample rows are arranged as follows from left to right with ascending numbers. Row 1: 100 bp size ladder; row 2: φX174/HaeIII; rows 3-11: clones 1-9. This shows that the library is not dominated by a few clones.

### Selection of Specific Phage Antibodies from the Cumulative Library

A combination of phosphatidylserine and beta-2-glycoprotein 1 (GP1) was chosen as the antigen for the selection. It is known that autoantibodies against this combinational epitope are associated with disorders of syncytial fusion. The autoantibodies are characterized by binding to phosphatidylserine, which appears at the outside of the cells shortly before fusion, in the presence of GP1.

To obtain antibodies having a specificity comparable to that, the following selection strategy was adopted:

1) 0.1 μg of phosphatidylserine was dissolved in 100 μl of ethanol and bound in an ELISA well at 37° C.

2) The well was washed three times with bidistilled water for 5 min.

3) The well was incubated (30 minutes at 37° C.) with phosphate-buffered (pH 7.2) isotonic saline (containing 10% fetal calf serum). At the same time, the phages to be selected were also incubated with the same solution under the same conditions in a well containing no phosphatidylserine. The calf serum serves both as a blocking agent and as a source of GP1. Human and bovine GP1s are highly homologous proteins which are both recognized by the human autoantibodies.

4) The phage suspension was added to the well containing phosphatidylserine and incubated at 37° C. for 1 h.

5) After this incubation time, the phage-containing suspension was removed from the well and washed at least three times with phosphate-buffered (pH 7.2) isotonic saline (containing 10% fetal calf serum) for 5 min each to remove non-specifically binding phages and those phages which react with GP1 alone.

6) Binding phages were eluted with glycine buffer (pH 2.2), the solution was neutralized, and the phages obtained were used for infection of E. coli XL-1 Blue. After amplification of the eluted phages in E. coli over night, the phages produced were harvested and recycled to step 1. Steps 1 to 5 were thus performed a total of five times in succession. The stringency of the washing steps in step 5 was increased in every cycle by extending the washing time.

From the phage population recovered after the last cycle, 15 arbitrary clones were isolated, and monoclonal phage suspensions were prepared by culturing over night.

The isolated clones and the unselected phages of the cumulative library were tested for reactivity with phosphatidylserine in a phage ELISA.

Thus, steps 1 to 5 as described above were performed. Subsequently, the wells were incubated with a commercial mouse antibody against the phages and with a peroxidase-coupled antibody against mouse immunoglobulins. The peroxidase was detected by a chromogenic reaction, and the dye quantity was determined by photometry in an ELISA reader. A higher absorption indicates a higher number of binding phages. The results for 15 clones and the cumulative library are summarized in Table 3. Among the 15 clones, there are some (1, 2, 4, 8, 10 and 15) which bind very much more strongly to phosphatidylserine than the phages of the cumulative library do. This is demonstrated by the successful enrichment and selection of specifically binding antibodies from the cumulative library.

### Fusion Assay

The thus cloned phage antibodies against GP1 and PHS (phosphatidylserine) were tested in a fusion assay. The object of this assay was to establish the functional properties of a phage antibody which need not necessarily be in agreement with its binding strength to PHS or GP1. Thus, cells of the human trophoblast cell line AC-1M17 are used which have a spontaneous fusion rate of about 20-30% immediately after inoculation into a culturing vessel.

Cells of this cell line are first proliferated to a sufficient number and then divided into two populations which are stained with different commercially available fluorescent dyes (DiI and DiO, staining according to the supplier's directions, supplied by Molecular Probes Europe B. V., Leiden, NL). After the cells have been thoroughly washed (5×10 minutes) to wash off any adhering residual dye which has not been integrated in the membrane, the two cell populations are mixed and commonly incubated at a low cell density in Hams F12 (10% fetal calf serum and antibiotics) under standard culturing conditions (5% carbon dioxide, 38° C., over 90% humidity) for 24 hours. After this culturing phase, the cells were detached from the bottom of the culture dish using trypsin and suspended, and a flow cytometer was used to determine how many of the cells present have acquired double fluorescence by fusion. FIG. 3 shows an example of such an evaluation of a fusion assay in a flow cytometer. Under control conditions (FIG. 3a, in the presence of the non-specific phage M13), the spontaneous fusion rate is at 19%, while the GP1-specific phage clone 2 results in a decrease of the fusion rate to 11%.

### TABLES

Table 1: Sequences of the primers for the amplification of Vh and Vk and the overlap extension PCR

!Acronym? PCR: Vh and Vk

| | |
|---|---|
| | a) Vk |
| CSCVK | 5′GTGGCCCAGGCGGCCCTGACTCAGCCGTCCTCGGTGTC3′ |
| CKJo-B | 5′CGAAGATGTAGAGGACTGACCTAGGACGGTCAGG3′ |
| | b) Vh |
| CSVHo-F | 5′GGTCAGTCCTCTAGATCTTCCGCCGTGACGTTGGACGAG |
| | 3′ |
| CSCG-B | 5′CTGGCCGGCCTGGCCACTAGTGGAGGAGACGATGACTTCG |
| | GTCC3′ |
| | Overlap extension PCR |
| CSC-F | 5′GAGGAGGAGGAGGAGGAGGTGGCCCAGGCGGCCGTGACTC |
| | AG3′ |
| CSC-B | 5′GAGGAGGAGGAGGAGGAGGAGCTGGCCGGCCTGGCCACTA |
| | GTGGAGG3′ |

TABLE 2
Designation, antigen and size (number of transformants)
per library

| Phage display | | |
|---|---|---|
| library (PDL) | Antigen | Transformants |
| PDL 1-4 | homogenizate of mature human | 1.2 · 10⁹ |
| | placenta | |
| PDL 5 | living cells, trophoblast cell line | 10⁷ |
| | ACt-1 | |
| PDL 6 | living cells, trophoblast cell line | 9.1 · 10⁷ |
| | Jeg-3 | |
| PDL 7 | living cells, trophoblast cell line | 8.6 · 10⁷ |
| | AC-1M88 | |
| PDL 8 | primary villous trophoblast, | 1.8 · 10⁹ |
| | freshly isolated from a mature | |
| | placenta | |
| PDL 9 | primary invasive trophoblast, | 3.8 · 10⁷ |
| | freshly isolated from fetal | |
| | membranes of a mature | |
| | placenta | |
| PDL 10 | living cells, trophoblast cell line | 3.1 · 10⁷ |
| | AC-1M32 | |
| PDL 11 | living cells, trophoblast cell line | 2.1 · 10⁷ |
| | AC-1M59 | |
| PDL 12 | purified human beta-2- | 2 · 10⁸ |
| | glycoprotein I | |
| PDL 15 | homogenizate of rat placenta | 4.7 · 10⁷ |
| PDL (cumulative | mixture of all phage libraries | total: 4 · 10⁹ |
| library) | | |

TABLE 3
Comparison of the 15 clones with the initial library in
phage ELISA

| | | | Control |
|---|---|---|---|
| Phage | Number of phages | PHS-coated well | well with no |
| population | applied per well | (absorption) | PHS (absorption) |
| Clone 1 | 10⁹ | 1.04 | 0.34 |
| Clone 2 | 10⁹ | 0.99 | 0.28 |
| Clone 3 | 10⁹ | 0.43 | 0.29 |
| Clone 4 | 10⁹ | 0.99 | 0.24 |
| Clone 5 | 10⁹ | 0.33 | 0.27 |
| Clone 6 | 10⁹ | 0.78 | 0.22 |
| Clone 7 | 10⁹ | 0.34 | 0.29 |
| Clone 8 | 10⁹ | 0.96 | 0.27 |
| Clone 9 | 10⁹ | 0.62 | 0.32 |
| Clone 10 | 10⁹ | 1.22 | 0.34 |
| Clone 11 | 10⁹ | 0.28 | 0.34 |
| Clone 12 | 10⁹ | 0.67 | 0.30 |
| Clone 13 | 10⁹ | 0.54 | 0.33 |
| Clone 14 | 10⁹ | 0.87 | 0.29 |
| Clone 15 | 10⁹ | 1.21 | 0.34 |
| Cumulative | 10⁹ | 0.31 | 0.26 |
| library | | | |

PHS is the abbreviation of phosphatidylserine.

1. A method for the preparation of specific monoclonal immunological binding molecules having binding capacity to mammal epitopes, comprising the following steps:
a) isolation of structures containing said epitopes to obtain an epitope preparation;
b) immunization of non-mammals with the epitope preparation to obtain an immune response;
c) immortalization of the immune response to obtain a library of immunological binding molecules;
d) selection of the immunological binding molecules by means of the epitopes to obtain specific monoclonal immunological binding molecules.

2. The method according to claim 1, characterized in that said monoclonal immunological binding molecules are antibodies or antibody fragments, especially single-chain antibodies (scFv).

3. The method according to claim 1, characterized in that:
said epitopes are expressed on the surfaces of cells, especially of trophoblasts or tumor cells; or
said epitopes are involved in cell-virus fusion; or
said epitopes are derived from endogenous antibodies.

4. The method according to claim 1, characterized in that the structures containing said epitopes are immunological binding molecules, especially antibodies or antibody fragments.

5. The method according to claim 4, characterized in that the structures containing said epitopes are immunological binding molecules obtained by a method according to claim 1.

6. The method according to claim 1, characterized in that said epitopes are oncofetal epitopes or epitopes involved in the syncytial fusion of trophoblasts.

7. The method according to claim 1, characterized in that said mammal species is selected from the group consisting of Homo sapiens, pets such as dogs and cats, and pests such as mice and rats.

8. A method for the identification of low molecular weight substances mimicking mammal epitopes, wherein the specific monoclonal immunological binding molecules obtained by the method according to claim 1 are used for identifying, in a library of low molecular weight substances, those having a high binding affinity.

9. The method according to claim 8, characterized in that said library of low molecular weight substances contains peptides.

10. The method according to claim 8, characterized in that said library of low molecular weight substances is a phage library.

11. An immunological binding molecule obtainable by a method according to claim 1.

12. A diagnostic agent containing at least one immunological binding molecule according to claim 11.

13. A low molecular weight substance obtainable by a method according to claim 8.

14. The low molecular weight substance according to claim 13, characterized by being a peptide.

15. The low molecular weight substance according to claim 14, characterized in that said peptide comprises from 7 to 15 amino acids.

16. A medicament containing a low molecular weight substance according to claim 13.

17. Use of the low molecular weight substance according to claim 13 for the preparation of a vaccine for contraception, for tumor treatment or for the treatment of infections.

18. A method for the preparation of specific single chain antibody fragments (scFV) having binding capacity to trophoblastic epitopes of mammals, comprising the following steps:
e) isolation of trophoblasts to obtain a trophoblast preparation;
f) immunization of chickens with the trophoblast preparation to obtain an immune response;
g) immortalization of the immune response to obtain a phage library;
h) selection of the single chain antibody fragments by means of trophoblasts to obtain specific single chain antibody fragments.

19. A method for the identification of peptides which inhibit the fusion of trophoblasts, wherein the specific single chain antibody fragments obtained by the method according to claim 18 are used for identifying, in a library of peptides, those having a high binding affinity.
